(19) United States
(12) Patent Application Publication
(10) Pub. No.: US 2025/0176936 A1
(43) Pub. Date: Jun. 5, 2025

(54) SYSTEM AND METHOD FOR INTRAOPERATIVE ANATOMICAL IMAGING

(71) Applicant: ORTHOSOFT ULC, Montreal (CA)

(72) Inventors: Louis-Philippe AMIOT, Montreal (CA); Karine DUVAL, Montreal (CA)

(21) Appl. No.: 18/962,555

(22) Filed: Nov. 27, 2024

Related U.S. Application Data
(60) Provisional application No. 63/605,716, filed on Dec. 4, 2023.

(51) Int. Cl. A61B 8/14; A61B 8/00

(52) U.S. Cl. CPC A61B 8/14; A61B 8/40; A61B 8/4209

(57) ABSTRACT

An intraoperative anatomical imaging system may have a support panel having a support surface configured for supporting a patient, the support surface defining a plurality of apertures distributed thereon. A peg(s) has an extremity insertable in one of the apertures such that when the extremity is inserted in the aperture the at least one peg is secured to the support surface, the at least one peg configured to position the patient on the support surface. An ultrasound probe unit may be emitting ultrasound signals successively towards different portions of an anatomical feature of the patient, measuring echo signals returning from the portions of the anatomical feature and generating respective imaged echo datasets. A controller is communicatively coupled to the ultrasound probe, the controller having a processor and a memory having stored thereon instructions that when executed by the processor perform the step of: registering the imaged echo datasets in an imaging system; and obtaining an ultrasound image of the anatomical feature based on said registering the imaged echo datasets in the imaging system. The ultrasound probe unit is secured to at least one of the at least one peg and the support surface such that the ultrasound image of the anatomical feature is obtainable when the patient is positioned on the support surface.

### CROSS-REFERENCE TO RELATED APPLICATION

The present application claims the priority of U.S. Patent Application No. 63/605,716, filed on Dec. 4, 2023, the content of which is incorporated herein by reference.

### TECHNICAL FIELD

The present disclosure relates to the field of computer-assisted surgery, and more specifically, to anatomical feature imaging and tracking in computer-assisted surgery (CAS) systems.

### BACKGROUND

Computer-assisted surgery (CAS) or image-guided surgery improves the quality of surgical procedures by increasing the accuracy at which the surgeon can operate. In CAS, tracking enables operators to have access to navigation data giving precise orientational and/or positional values to relate instruments to bones in real time. Yet, in some cases, imaging the area surrounding anatomical features may prove challenging due to the nature of the surgery.

For example, the pelvis remains for the most part concealed under soft tissue. As such, there are challenges in correctly tracking the pelvis, notably due to movements that may occur intraoperatively. In response to these issues, surgical tools such as surgical pegboard positioners, which maintains the patient in position during the surgery, were developed. Compatibility between surgical pegboard positioners and CAS is, however, lacking.

There thus remains room for improvement.

### SUMMARY

In a first aspect, there is provided an intraoperative anatomical imaging system comprising: a support panel having a support surface configured for supporting a patient, the support surface defining a plurality of apertures distributed thereon; at least one peg having an extremity insertable in one of the apertures such that when the extremity is inserted in the aperture the at least one peg is secured to the support surface, the at least one peg configured to position the patient on the support surface; at least one ultrasound probe unit; and a controller being communicatively coupled to the ultrasound probe, the controller having a processor and a memory having stored thereon instructions that when executed by the processor perform the step of: controlling the at least one ultrasound probe unit to emit ultrasound signals towards an anatomical feature of the patient, measuring echo signals returning from the anatomical feature, and generating an ultrasound image of the anatomical feature based on the echo datasets; wherein the at least one ultrasound probe unit is secured to at least one of the at least one peg and the support surface such that the ultrasound image of the anatomical feature is obtainable when the patient is positioned on the support surface.

Further in accordance with the first aspect, for instance, the ultrasound probe comprises a plurality of ultrasound probe units mounted to at least one of the support surface and the at least one peg.

Still further in accordance with the first aspect, for instance, at least one of the support surface and the at least one peg is configured to propagate the ultrasound signals and the echo signals.

Still further in accordance with the first aspect, for instance, the ultrasound probe unit is integrated inside at least one of the at least one peg.

Still further in accordance with the first aspect, for instance, the ultrasound probe unit is on an underside of the support surface.

Still further in accordance with the first aspect, for instance, generating an ultrasound image of the anatomical feature includes generating an anatomical feature model of the anatomical feature based at least on the echo datasets.

Still further in accordance with the first aspect, for instance, said generating the anatomical feature model includes merging the ultrasound image with a reference model of the anatomical feature.

Still further in accordance with the first aspect, for instance, said reference model is obtained from a magnetic resonance imaging scan.

Still further in accordance with the first aspect, for instance, said reference model is obtained from a bone atlas of models.

Still further in accordance with the first aspect, for instance, a coordinate tracking unit may track coordinates of at least one of the support panel, the at least one peg and the ultrasound probe unit.

Still further in accordance with the first aspect, for instance, the controller is communicatively coupled to the coordinate tracking unit and the memory has stored thereon instructions that when executed by the processor perform the steps of: generating corresponding coordinate datasets, registering the corresponding coordinate datasets in a common coordinate system, and tracking a position and orientation of the anatomical feature based on said registering the echo datasets in the common coordinate system.

Still further in accordance with the first aspect, for instance, the at least one ultrasound probe unit includes a first ultrasound probe unit configured to emit ultrasound signals towards the anatomical feature of the patient, and a second ultrasound probe unit for measuring echo signals returning from the anatomical feature.

In accordance with a second aspect of the present disclosure, there is provided an intraoperative anatomical imaging system comprising: a support panel having a support surface configured for supporting a patient, the support surface defining a plurality of apertures distributed thereon; at least one peg having an extremity insertable in one of the apertures such that when the extremity is inserted in the aperture the at least one peg is secured to the support surface, the at least one peg configured to position the patient on the support surface; and at least one ultrasound probe unit secured to the at least one peg the ultrasound probe unit configured for emitting ultrasound signals towards different portions of an anatomical feature of the patient, and a second ultrasound probe unit for measuring echo signals returning from the portions of the anatomical feature, whereby an ultrasound image of the anatomical feature is obtainable when the patient is positioned on the support surface.

Further in accordance with the second aspect, for instance, the at least one ultrasound probe unit is integrated inside at least one of the at least one peg.

Still further in accordance with the second aspect, for instance, the at least one ultrasound probe unit includes another ultrasound probe unit on an underside of the support surface.

Still further in accordance with the second aspect, for instance, the at least one ultrasound probe unit includes a first ultrasound probe unit configured to emit ultrasound signals towards the anatomical feature of the patient, and a second ultrasound probe unit for measuring echo signals returning from the anatomical feature.

In accordance with a third aspect of the present disclosure, there is provided an intraoperative anatomical imaging system comprising: a processor; and a memory having stored thereon instructions that when executed by the processor perform the step of: emitting ultrasound signals towards an anatomical feature of the patient, from a peg projecting from a support panel, with the anatomical feature captive against the peg and support panel, measuring echo signals returning from the anatomical feature, and generating and outputting an ultrasound image of the anatomical feature based on the echo datasets.

Further in accordance with the third aspect, for instance, generating an ultrasound image of the anatomical feature includes generating an anatomical feature model of the anatomical feature based at least on the echo datasets.

Still further in accordance with the third aspect, for instance, said generating the anatomical feature model includes merging the ultrasound image with a reference model of the anatomical feature.

Still further in accordance with the third aspect, for instance, said reference model is obtained from a magnetic resonance imaging scan.

Still further in accordance with the third aspect, for instance, said reference model is obtained from a bone atlas of models.

### BRIEF DESCRIPTION OF THE DRAWINGS

Further features and advantages of the present disclosure will become apparent from the following detailed description, taken in combination with the appended drawings, in which:

FIG. 1 is a perspective view of an intraoperative anatomical imaging system, in accordance with one or more embodiments;

FIG. 2 is a perspective view of the system of FIG. 1 in which a patient is positioned on a support plate, in accordance with one or more embodiments;

FIG. 3 is a view schematizing an ultrasound imaging system, in accordance with one or more embodiments;

FIG. 4 is a view schematizing a system including an ultrasound probe, a controller and a coordinate tracking unit, in accordance with one or more embodiments;

FIG. 5 is a flow chart of an example of a method for imaging an anatomical feature in computer-assisted surgery;

FIG. 6 is a flow chart of an example of a method for tracking a position and orientation of an anatomical feature in computer-assisted surgery; and

FIG. 7 is a block diagram of an example computing device, in accordance with an illustrative embodiment.

Many further features and combinations thereof concerning the present improvements to those skilled in the art following a reading of the instant disclosure.

### DETAILED DESCRIPTION

Described herein is a system for intraoperative anatomical imaging having an ultrasound imaging system coupled to a surgical pegboard positioner. Such system can achieve precise imaging of areas of operation without relying on complex and cumbersome equipment that may limit the movement of the surgeon. Thus, the present technology provides patient stability and accessibility to the surgeon in operation, while also providing an operator with ultrasound images of anatomical features (such as bones and soft tissue).

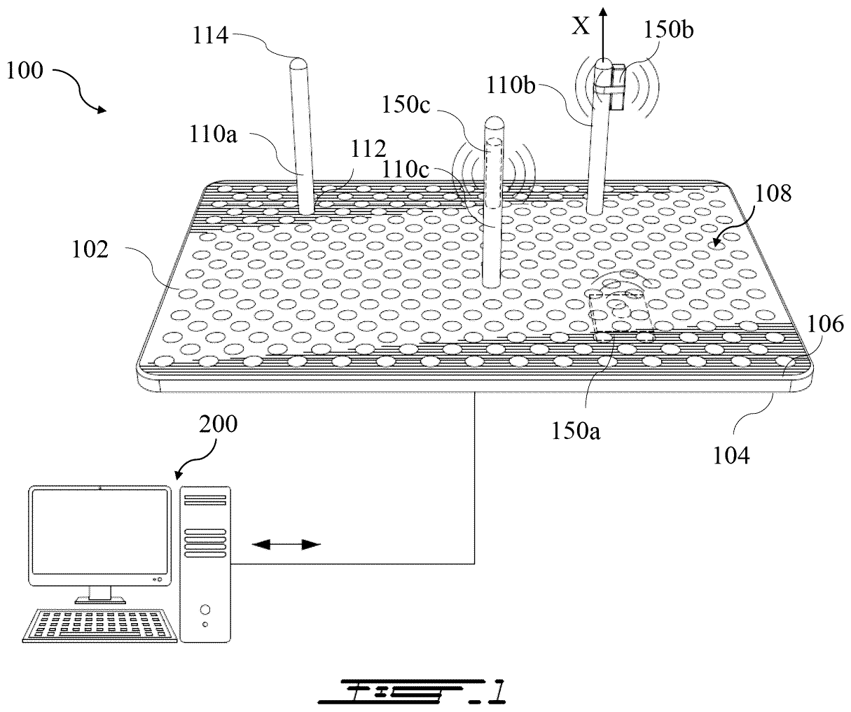

Referring to the drawings and more particularly to FIG. 1, there is shown a perspective view of an intraoperative anatomical imaging system 100. The system 100 includes a support panel 102 having a bottom surface 104 and a top surface 106. The top surface 106 defines a plurality of apertures 108 extending at least partially through the support panel 102. The system 100 further includes a plurality of pegs 110a,b,c each having a first end 112 and a second end 114, the first end 112 of the pegs 110a,b,c being insertable in one of the plurality of apertures 108. Furthermore, the system 100 includes one or more ultrasound probes, shown as 150a, 150b and 150c communicatively coupled to a controller 200 for imaging anatomical features. The system 100 may have one or more of the types of ultrasound probes 150a, 150b, 150c, with the possibility of having multiple ones of a same type of ultrasound probes 150a, 150b, 150c.

The support panel 102 is configured for supporting a patient. It is thus understood that the size and shape of the support panel 102 is adapted so that a person may lay thereon. For example, the support panel 102 may be part of an operating room table, or may be laid on an operating room table or like patient-receiving surface. In an exemplary embodiment, the support panel 102 has a length of 120 cm (±20 cm), a width of 50 cm (±10 cm) and a thickness of 3 cm (±2 cm), these dimensions being given as an example. The support panel 102 mat be outside of these dimensional ranges. These dimensions may be suitable when the support panel 102 is laid onto another surface, as these dimensions may be smaller than the height of an adult patient. In some embodiments, the support panel 102 includes separate panels that may be attachable with one another, in a modular configuration. While the depicted support panel 102 has a rectangular prism shape with rounded corners, it will be appreciated that the shape of the support panel 102 may vary in accordance with the embodiment. In other embodiments, support legs (not depicted) are attached to the bottom surface 104 of the support panel 102. In other embodiments, the support panel 102 includes rail clamps (not depicted) attachable to a support rail.

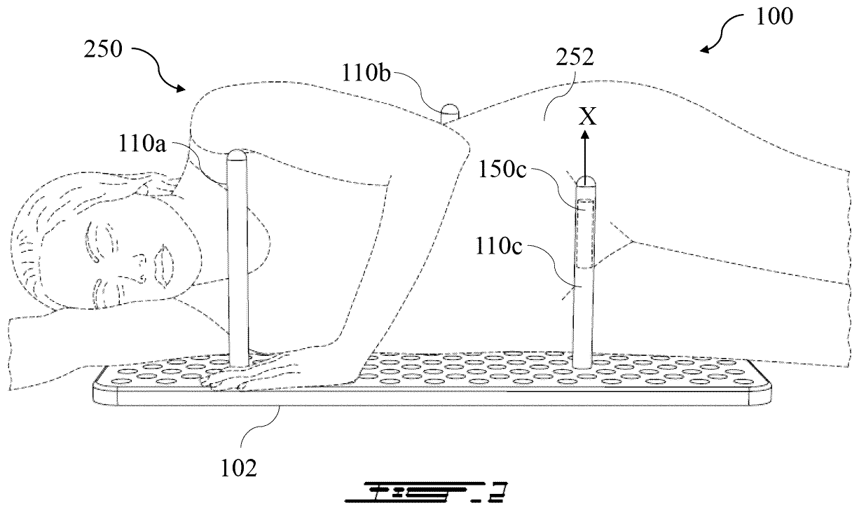

The plurality of apertures 108 are configured to receive the pegs 110 such that the pegs 110a,b,c are secured to the support panel 102 when inserted in the apertures 108. In some embodiments, each aperture 108 and the pegs 110a,b,c define matching components interlockable with each other, such as in a mating arrangement in which the pegs 110a,b,c penetrate into selected ones of the apertures 108. The plurality of apertures 108 may be distributed on the top surface 106 of the support panel 102 in a uniform and/or periodic pattern, though this is optional. Each aperture 108 may extend fully or partially through the support panel 102. There may be substantially more apertures 108 than pegs 110, with the pegs 110 positionable at desired locations relative to the patient, to hold the patient in position that is selected by the operator. For example, the patient may be in lateral decubitus as shown in FIG. 2, with the patient's pelvis held generally stationary by the pegs 110. The patient could be in other positions, such as supine decubitus, for example.

The pegs 110a,b,c are designed to securely hold the patient in place, thus generally preventing or limiting movements during the surgical procedure. The pegs 110a,b,c generally have a rod shape with a rounded second end 114. In some cases, the pegs 110a,b,c may be hollow. The diameter of the pegs 110a,b,c is chosen to tightly fit in the apertures 108. The length of the pegs 110a,b,c may vary according to the type of surgery and the size of the patient. In some embodiments, the length of the pegs varies between 20 cm and 40 cm. In some embodiments, the pegs 110a,b,c are covered with a resilient cover (not depicted) to provide comfort to the patient.

As depicted in FIG. 1, an ultrasound probe 150b is mounted on one peg 110b on the outer surface thereof near the second end 114. The ultrasound probe 150b may be attached via a fastener or bounded to the outer surface of the peg 110b. In some embodiments, the ultrasound probe 150b is built-in on the outer surface of the peg 110b. As another embodiment, that may be used in conjunction with or alternative to the ultrasound probe 150b, an ultrasound probe 150c may also be included within a peg 110c. As such, the patient would not be in direct contact with the ultrasound probe 150c, but in a fixed relation related to the ultrasound probe 150c. It will be appreciated that the ultrasound probes 150b,c may be positioned at any given portion of the respective peg 110b,c along an elongation axis X. In some embodiments, the support panel 102 includes an ultrasound probe(s) 150a configured to emit ultrasound signals outward of the top surface 106. While FIG. 1 shows one example of each probe 150a, 150b, 150c, there may be multiple ones of one or more of such probe types, such as in each peg 110, or to cover the whole top surface 106 of the support panel 102.

The support panel 102 and/or the pegs 110a,b,c may include electrical connections to communicatively couple the controller 200 with the ultrasound probe 150a,b,c, such that the positioning of the pegs 110a,b,c in the apertures 108 causes activation of any active probe therein. The support panel 102 and/or the pegs 110a,b,c may be made of metals such as aluminum and steel, of plastic and/or combination thereof, and are generally made of a lightweight material. The support panel 102 and the pegs 110a,b,c are usually rigid to withstand the pressures applied during surgery. In some embodiments, the support panel 102 and/or the pegs 110a,b,c are made at least partially of a material that propagates ultrasounds. As such, the ultrasound probes 150a,b,c do not need to be in contact or next to the anatomical feature in order to obtain an ultrasound image thereof. While the three ultrasound probes 150a,b,c and three pegs 110a,b,c are depicted on FIG. 1, it will be understood that the number of ultrasound probes 150a,b,c and pegs 110a,b,c may each vary according to the embodiment. In a variant, the apertures 108 each have a location identity, i.e., the apertures 108 have a geometrical position in the support panel 102, which position is known by the controller 200. Therefore, when one of the apertures 108 receives one of the pegs 110 with probe 150b or 150c, the controller 200 knows where the ultrasound emission is generated from. The same notion applies to the probe(s) 150a in the support panel 102. It can be said that the support panel 102 and pegs 110 are pre-calibrated. Therefore, when a model of an anatomical feature is generated, this geometrical position data may be used to combine the readings from the various probes 150a,b and/or c. The calibration may also be done intraoperatively, for example using the optical tracking system described hereinafter.

As shown, the controller 200 may be part of a computer, or may be implemented in the form of a personal computer, a laptop, a tablet, a server, etc., that may be dedicated to anatomical imaging and surgical flow assistance. The controller 200 may be configured for executing computer-readable program instructions suitable for the processing of datasets related to anatomical imaging. The program instructions may originate from a medium either remote from the controller 200 or directly connected thereto. Among possible implementations, the program instructions may be stored on a non-transitory computer-readable medium which may be communicatively coupled to the controller 200. The controller 200 will be described further below.

Now referring to FIG. 2, there is shown a perspective view of the system 100 of FIG. 1 in which a patient 250 is positioned on a support panel 102 in lateral decubitus, for example for operation on the left acetabulum, such as in the context of partial hip replacement, hip resurfacing, total hip replacement, etc. Three pegs 110a,b,c are inserted in the support panel 102 in a configuration favorable for maintaining the patient 250 in a lateral decubitus (or decubitus) position. As such, the peg 110a is provided on the support panel 102 to contact the thorax of the patient 250, the peg 110b is provided on the support panel 102 to contact the lumbar region of the patient 250 and the peg 110c is provided on the support panel 102 to contact the hips of the patient 250, such that the patient is held captive. In some embodiments, a gel cover (not depicted, but known as ultrasound gel or coupling gel) is provided on the support panel 102 to assist in the propagation of the ultrasound signals from the probe 150a, if present. Moreover, even though the peg 110c is depicted with the probe 150c, the probe or another probe could be in the peg 110b.

An ultrasound probe 150c is attached to the peg 110c in order to image an anatomical feature 252 of the patient 250, i.e., it is within the peg 110c, if the probe is of the type shown as 150c, as 150b could also be used. In this case, the anatomical feature 252 may be, for instance, the pelvis. The ultrasound probe 150c may be positioned along an elongation axis X in order to be aligned with the lateral axis of the pelvis, though this is optional. The ultrasound probe 150c may be oriented towards the anatomical feature 252 such that ultrasound signals are emitted in the direction of the anatomical feature. In some embodiments, the outer surface of the peg 110c comprises an alignment marker or like clocking feature (not depicted) radially aligned with the direction at which the ultrasound are emitted from the ultrasound probe 150c. The alignment marker may be, for instance, a dot aligned with the center of the ultrasound probe 150c or a line extending at least partially along the elongation axis X, a key or keyway. In one embodiment, the anatomical feature 252 is the pelvis of the patient 250. It will be understood that ultrasound probe 150 may be provided in the pegs 110b,c and/or in the support panel 102, such that the combined readings of the various probes can contribute to generating a model of the pelvis or other target anatomical feature.

In other embodiments, the patient 250 may be positioned on the support panel 102 in one of a supine decubitus position, a prone position and a Fowler's position. The configuration of the support panel 102 and the pegs 110a,b,c may be provided and positioned accordingly.

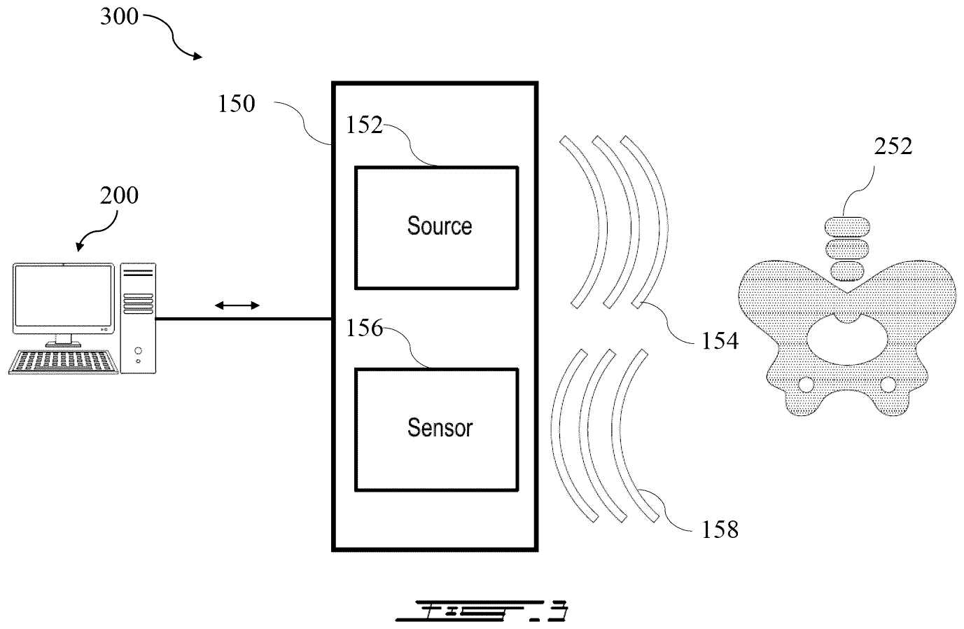

FIG. 3 is a view schematizing an ultrasound imaging system 300 having one or more ultrasound probe(s) 150 and a controller 200 for imaging an anatomical feature 252. The ultrasound probe 150 may correspond to the one or more of the ultrasound probes 150a,b,c of the system 100.

In some embodiments, the ultrasound probe(s) 150 is(are) used to produce a signal indicative of at least one spatial and/or dimensional characteristic relating to biological tissue. According to conventional ultrasound-based detection principles, the ultrasound probe 150 includes an ultrasound emitter, in this case the source 152 (e.g., incorporating a pulse generator), that may be used to cast a sound wave 154 and, upon an object being located within range of the source 152, a sensor 156 senses an echo 158 of the sound wave 154 reflected by the object. In some embodiments, the source 152 and the sensor 156 may be separate from one another. However, in some other embodiments, the source 152 and the sensor 156 may be combined to one another in an ultrasound transducer performing both the ultrasound emission and the sensing functions, such as in the probe(s) 150 shown herein. The echo may materialize upon the sound wave travelling through a first medium, such as skin, reaching a second medium of greater density compared to that of the first medium, such as a bone. As the speeds at which the sound waves may travel through various media depend on the respective physical properties of such media, characteristics of the echo (e.g., time elapsed between emission of the sound wave and the sensing of the echo, intensity of the echo relative to that of the sound wave, etc.) may be used to derive certain characteristics of the media through which the echo has travelled. In some embodiments, the functions of both the source 152 and the sensor 156 are performed by one or more ultrasound transducer transducers. In some embodiments, the ultrasound transducer may have one or more piezoelectric crystals emitting ultrasound signals based on corresponding electrical signals, and/or generating electrical signals based on received ultrasound signals. Any suitable type of ultrasound transducers can be used including, but not limited to, piezoelectric polymer-based ultrasound transducers such as poly(vinylidene fluoride)-based ultrasound transducers, capacitive ultrasound transducers, microelectromechanical systems (MEMS) based ultrasound transducers and the like.

Per the present disclosure, namely, in the exemplary case of orthopedic surgery for instance, the ultrasound imaging system 300 may be configured to produce a signal indicative of a detailed spatial relationship between the ultrasound probe(s) 150 and the anatomical feature 252, and also between constituents of the anatomical feature 252 such as soft tissue (e.g., skin, flesh, muscle, ligament) and bone. Resulting datasets may include measurements of a distance between contours associated to the anatomical feature, such as an epithelial contour associated to skin and a periosteal contour associated to the bone. The resulting datasets may also include measurements of thicknesses, surfaces, volumes, medium density and the like. Advantageously, updated signal production via the ultrasound imaging system 300 and ad hoc, quasi-real-time processing may produce datasets which take into account movement and/or deformation of one or more of the constituents of the anatomical feature 252. The ultrasound imaging system 300 may also include a dedicated computing device configured for conditioning and/or digitizing the signal.

In some implementations, the ultrasound imaging system 300 may be suitable for producing a signal indicative of surfacic, volumetric and even mechanical properties of the anatomical feature 252. This may be achieved, for instance, by way of a multi-planar ultrasound system capable of operating simultaneously along multiple notional planes that are spaced and/or angled relative to one another, coupled to a suitably configured controller 200. Further, it is contemplated that other types of imaging systems, such as an optical coherence tomography (OCT) system, may be used in combination with the ultrasound imaging system 300. The type of additional imaging system may be selected, and combined with other type(s) as the case may be, to attain certain performance requirements in terms of effective range, effective depth, signal-to-noise ratio, signal acquisition frequency, contrast resolution and scale, spatial resolution, etc., among other possibilities.

In some embodiments, the ultrasound imaging system 300 may be provided in an ultrasound transmission tomography configuration in which one peg 110 may include a source 152 and another peg may include a sensor 156, such as 110b and 110c in FIG. 2. In this case the pegs 150 may be placed on each sides of the patient so that the anatomical feature 252 lays therebetween. In operation, the ultrasound signals 154 are emitted by the source 152 in one peg 110 and the echo signals 158 are sensed by the sensor 156 in the other peg. In a variant, each peg 110 (or two or more peg 110) and the support panel 102 include a probe 150, and the anatomical feature 252 is imaged by sequentially emitting ultrasound signals from one probe 150 and measuring the transmitted probed signal using the other probes 150. It will be understood that the ultrasound signals emitted by each probe 150 may be measured by other probes 150, according to the configuration and the embodiment.

In an embodiment, the ultrasound probe 150 include one or more transducers that emit an ultrasound wave and measure the time it takes for the wave to echo off of a hard surface (such as bone) and return to the face(s) of the transducer(s). In order to self-calibrate for the patient's individual speed of sound, some transducers are positioned very accurately relative to others and as one emits waves, others listen and can compute the speed of sound based on well-known relative geometric positioning. Using the known speed of the ultrasound wave travelling through a bodily media, the time measurement is translated into a distance measurement between the ultrasound probe 150 and the bone located below the outer-skin surface. The transducers in the ultrasound probe 150 may be single-element or multi-element transducers, or a combination of both. For example, the probe 160 may have multiple elements arranged in a phased array, i.e., phased-array ultrasound probe 150, having the capacity of performing multi-element wave generation for sound wave direction control and signal reconstruction. In some embodiments, the ultrasound probe 150 has a single ultrasound transducer operating in a phased-array arrangement. When sensors are not rigidly linked to others, the relative position can be found with self-location algorithms, or using the anatomical feature as reference, especially if the anatomical feature is generally immobile during the procedure. Therefore, the ultrasound probe 150 produces signals allowing local image reconstruction of the anatomical feature 252. The ultrasound probe 150 is configured to emit ultrasound signals successively towards different portions of the anatomical features 252. In some embodiments, the ultrasound signals may be successively steered from one portion to another. Alternatively or additionally, the ultrasound signals may be successively focused on the different portions of the anatomical feature 252. In one embodiment, the measurement is repeated at regular intervals. The measurements are constantly being transferred to the controller 200.

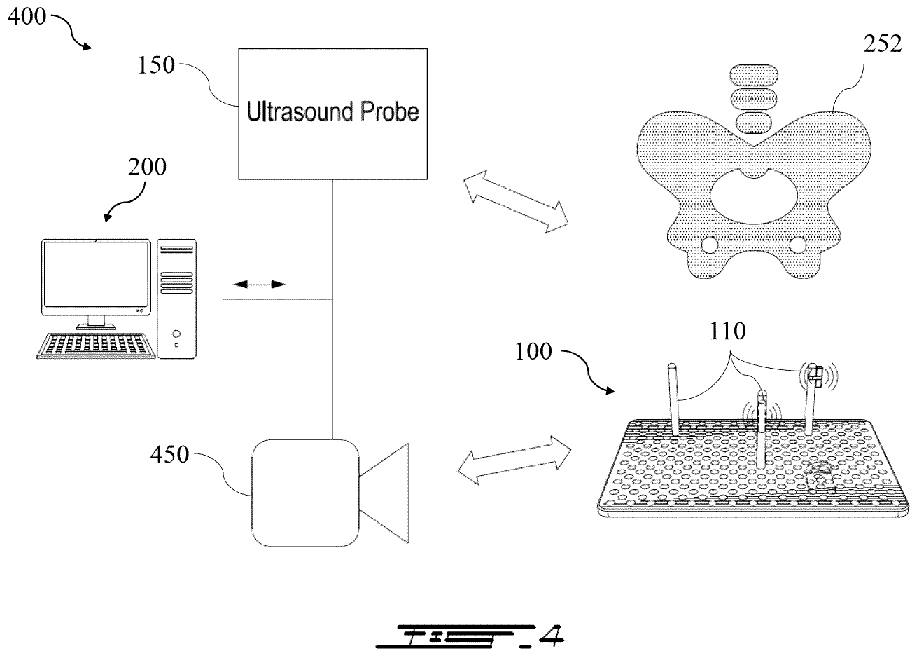

Now referring to FIG. 4, there is shown a view schematizing a system 400 comprising an ultrasound probe(s) 150, a controller 200 and a coordinate tracking unit 450 (also known as a tracker, e.g., Navitacker®) or depth camera that enable imaging and tracking of the position and orientation of an anatomical feature 252 of a patient placed on a support surface, such as the configuration presented in FIG. 2. It will be appreciated that the coordinate tracking unit 450 may be part of the system 300 in order to reproduce the system 400, given that the controller 200 is able to register imaged echo datasets in a common coordinate system and track a position and orientation of the anatomical feature 252.

The coordinate tracking unit 450 may include one or more coordinate tracking devices including, for instance, a tracker that tracks marker reference(s), such as a Navitracker® system, a depth camera that images the operative scene and has sufficient resolution to track objects. The coordinate tracking unit 450 can use either active or passive spatial references as markers of position and/or orientation. For example, as is known in the art, the coordinate tracking unit 450 may optically see and recognize retroreflective devices as reference markers, so as to track objects, for example tools and limbs, in six (6) degrees of freedom (DOFs), namely in position and orientation along the X, Y and Z axes. For example, the coordinate tracking unit 450 is a Navitracker® system that relies on optical tracking with trackable markers affixed to objects. Thus, the orientation and position of the limb in space can be determined using the information obtained from the spatial references, resulting in a corresponding dataset that is defined according to a corresponding coordinate system, which may in some cases be inherent to a reference marker placed near the anatomical feature 252. The coordinate tracking unit 450 may also include a dedicated computing device used to condition, digitize and/or otherwise process the signal produced by the camera. The coordinate tracking unit 450 may include a 3D depth camera as a possibility (e.g., a Kinect™), that may not require passive spatial references as markers of position and/or orientation. Other 3D cameras can be used in other embodiments. For instance, the coordinate tracking unit 450 may include conventional two-dimensional camera(s) (operated in mono- or stereo-vision configuration) operated with a shape recognition module which identifies, locates and processes two-dimensional identifiers (e.g., QR codes) as imaged in the two-dimensional images generated by the two-dimensional camera(s). In these embodiments, the shape recognition module can evaluate the distortion of the two-dimensional identifiers in the two-dimensional images (e.g., a square identifier becoming trapezoidal when bent) to retrieve three-dimensional model(s) of the two-dimensional identifiers and/or of the underlying anatomical feature.

In some embodiments, the support panel 102 and/or the pegs 110 may comprise or serve as support markers, and have markers thereon to serve as supports. Stated differently, the coordinate tracking unit 450 may optically track the pegs 110 to position them in space, with readings from probes 150b and/or 150c in the pegs 110 combined with the optical tracking. In such case, the coordinate tracking unit 450 may be calibrated using the support panel 102 and the pegs 110 configured for a given operation and for the size and shape of the patient, such that the position and orientation of the anatomical feature 252 may be tracked once the surgery starts.

In some cases, the position and orientation of other features may be tracking during surgery using the coordinate tracking unit 450, such as medical equipment, members of the surgical team, anatomical features of the patient that are not subject to the surgery and the like. In these cases, reference markers may be placed on the other features in order to improve tracking of their position and orientation.

In some embodiments, the position and the orientation of the anatomical feature 252, along with the ultrasound image thereof, may be displayed on a display device accessible to the surgeon during the operation.

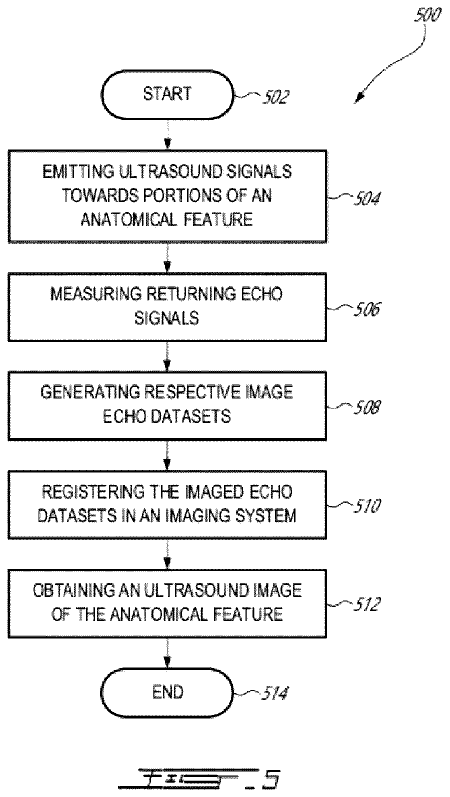

The flow chart of FIG. 5 depicts a method 500 for imaging an anatomical feature in computer-assisted surgery, which can be performed using the systems 100, 300 and 400 presented above. The method 500 starts at step 502.

At step 504, ultrasound signals are emitted towards portions of an anatomical feature 252, from probes 150 in the support panel 102 and/or from probes 150 in the pegs 110, such that the ultrasound signals are emitted from a structure supporting the patient, and holding a target anatomical feature 252 of the patient generally immobile. Thus the ultrasound signal emission is unique in its generation from a structural standpoint relative to the patient. In operation, one or more probes 150 may be placed in the support panel 102 beneath a chosen portion of the support panel 102 to receive the patient during the surgery. The position of each probe 150 in the pegs 110 and the orientation thereof may also be adjusted to focus the ultrasound signals towards the regions of interest, e.g. the anatomical feature 252 subject to the operation.

The ultrasound signals may be emitted in succession or simultaneously, for instance if from more than one probe 150. The ultrasound signals may be generated by a single transducer, a linear array of transducers or a 2D array of transducers. It will be appreciated that the frequency, the amplitude and the shape of the ultrasound signals may vary. For instance, the ultrasound signals may cover broad frequency ranges in order to map the anatomical feature 252 and distinguish the latter from other features in the same area. In other cases, the ultrasound signals include spectrally narrow signals that are configured for exciting specific portions of the anatomical feature 252. Generally, the ultrasound signals have a frequency between 1 megahertz and 18 megahertz. Also, the typical rate at which ultrasound signals are emitted generally is about 10 to 30 ultrasound signals per second.

In some embodiments, the ultrasound signals are chosen to minimize the body effects on the patient, such as tissue heating and cavitation. As such, the amplitude of the ultrasound signal may be chosen so that the body tissues are no warmed over 40 degrees Celsius when exposed to ultrasounds. In some cases, the temperature of the tissues of the anatomical feature 252 and/or the surroundings is measured by a sensor or a thermometer in order to detect and prevent harmful body effects.

At step 506, returning echo signals are captured and measured. The returning echo signals generally have a reduced amplitude compared to the ultrasound signals emitted towards the anatomical feature 252. This reduction may be caused in part by the absorption of the ultrasounds by the body when travelling to the object and when returning to the sensor. In a variant, the returning echo signals are captured from the support panel 102 and/or from probes 150 in the pegs 110, such that the ultrasound signals are captured from the structure supporting the patient, and holding the target anatomical feature 252 of the patient generally immobile. Again, the ultrasound signal capture is unique as it comes from a structural standpoint relative to the patient.

At step 508, respective image echo datasets are generated. The image echo datasets may include a plurality of ultrasound signals paired with a corresponding returning echo signals.

At step 510, the imaged echo dataset is registered in an imaging unit, which is configured for generating an ultrasound image of the anatomical feature 252. In some embodiments, an anatomical feature 252 model of the anatomical feature 252 is generated based at least on the imaged echo datasets.

In some embodiments, steps 504-510 are repeated so that enough pairs of ultrasound signals and returning echo signal are generated to recreate an image or a volume of the anatomical feature 252. As such, a 2D or 3D rasterization of the anatomical feature 252 may be processed by focusing the ultrasound signals on different portions of the anatomical feature 252. In other embodiments where a 2D array of transducers is used, a single pair of ultrasound signals and returning echo signals may be used to generate the ultrasound image. In some cases, a plurality of 2D images taken at different depths can be used to create a 3D ultrasound image of the anatomical feature 252.

At step 512, an ultrasound image of the anatomical feature 252 is obtained. To do so, the ultrasound signals are compared with their respective returning echo signals. In some embodiments, the time delay taken by the returning echo signal to return to the probe may be representative of the depth of the object that reflected the ultrasound signal. Also, the intensity of the returning echo signal may be representative of the surface uniformity and acoustic impedance, which may be correlated to the hardness, of the object reflecting the ultrasound signals. By analyzing the time delay and the intensity of each returning echo signals compared to their respective ultrasound signals, it is possible to obtain the ultrasound image. It will be appreciated that other types of signal analysis may apply. The ultrasound image may be in the form of a three-dimensional image of the anatomical feature 252.

In some embodiments, the imaged echo dataset is compared with a model of anatomical feature 252 (e.g., 3D model generated from pre-operative imaging) in order to improve the quality of the image, or to provide additional data to the tracking. For example, the model obtained pre-operatively may be merged with the ultrasound image or model. For example, a magnetic resonance imaging (MRI) scan may be used as a starting point for the model, and the imaged echo dataset is used when merging the model from MRI scan to the ultrasound measurement. Other imaging modalities could be used for generating a model, including radiography. Moreover, while the model of the anatomical feature 252 may be patient-specific, as it is generated from images of the patient, the system may retrieve a model from a bone atlas. The method 500 may end at step 514.

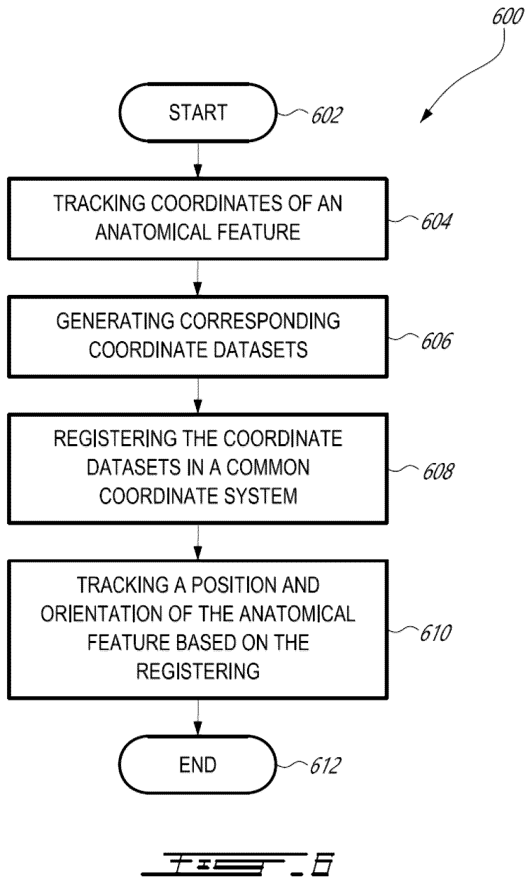

FIG. 6 is a flow chart of a method 600 for tracking a position and orientation of an anatomical feature 252 in computer-assisted surgery, which can be performed using the system 400 presented in FIG. 4. The method 600 starts at step 602.

At step 604, coordinates of an anatomical feature 252 are tracked. The tracking may be performed by a camera(s) positioned to capture the movements of the anatomical feature 252. For instance, the camera may be placed adjacent to or over the support surface on which the patient is placed during the surgery. Alternatively, the camera, or multiple cameras, may be placed on the walls of the operation room. In some embodiments, body markers are provided on the patient near the anatomical feature 252 in order to improve the precision of the tracking, and may be on some the pegs 110 and/or support panel 102.

Once the anatomical feature 252 has been identified by the camera, at step 606, a suited image recognition algorithm identifies the anatomical feature 252 in the captured image and generates a coordinate dataset corresponding to the position and the orientation of the anatomical feature 252 at the moment that the image has been captured. In some embodiments, the set of coordinates comprises six coordinates, namely the position and orientation along the X, Y and Z axes. Step 606 may include one or more of the steps of method 500, such that the coordinate datasets may be the result of the ultrasound imaging of method 500. Step 606 may thus occur prior to step 604.

In some embodiments, markers may be provided, such as on the patient, on the support surface and/or the pegs. Using extrapolation or interpolation, the position of the anatomical feature 252 may be obtained relatively to the marker(s), which has known position and orientation. In some cases, the coordinate of the support panel 102 and the pegs 110 are also tracked at step 604, which may serve as a mean to monitor intraoperative changes of the setup configuration. In this case, markers may be placed on the support panel 102 and the pegs 110 to track their coordinates.

At step 608, the coordinate datasets are registered in a common coordinate system, which is configured to track the position of the orientation of the anatomical feature 252 based on at least one coordinate dataset. In some cases, the coordinate datasets of the support panel and the pegs 150 are also registered in the common coordinate system.

At step 610, a position and orientation of the anatomical feature 252 are tracked based on the registering. In some cases, the position and orientation are also displayed on a display device. It will be understood that a plurality of coordinate datasets are required to track movements (i.e., a variation of the coordinates in time) of the anatomical feature 252. The position and orientation of the support panel may also be tracked at step 610, which enables to monitor the surgical setup during the surgery. By monitoring the surgical setup, it is possible to provide the position and orientation of each probe 150 in operation, which can be used in the modeling of the ultrasound image. The method 600 may end at step 612.

In some embodiments, the imaging system of method 500 and the common coordinate system of method 600 are included in the same processing device. While these systems are complementary for imaging and tracking an anatomical feature 252, communication between these systems may occur to improve performances. For instance, the position and orientation of the anatomical feature 252 may be provided to the imaging system in order to introduce spatial information in the image, which may help visualizing the anatomical feature 252. On the other hand, the image of the anatomical feature 252 may be provided to the common coordinate system to improve the identification of the anatomical feature 252 in the environment.

In a variant, the present disclosure pertains to an intraoperative anatomical imaging system comprising: a support panel having a support surface configured for supporting a patient, the support surface defining a plurality of apertures distributed thereon; at least one peg having an extremity insertable in one of the apertures such that when the extremity is inserted in the aperture the at least one peg is secured to the support surface, the at least one peg configured to position the patient on the support surface; and at least one ultrasound probe unit secured to the at least one peg the ultrasound probe unit configured for emitting ultrasound signals towards different portions of an anatomical feature of the patient, and a second ultrasound probe unit for measuring echo signals returning from the portions of the anatomical feature, whereby an ultrasound image of the anatomical feature is obtainable when the patient is positioned on the support surface.

In another variant, the present disclosure pertains to an intraoperative anatomical imaging system comprising: a processor; and a memory having stored thereon instructions that when executed by the processor perform the step of: emitting ultrasound signals towards an anatomical feature of the patient, from a peg projecting from a support panel, with the anatomical feature captive against the peg and support panel, measuring echo signals returning from the anatomical feature, and generating and outputting an ultrasound image of the anatomical feature based on the echo datasets.

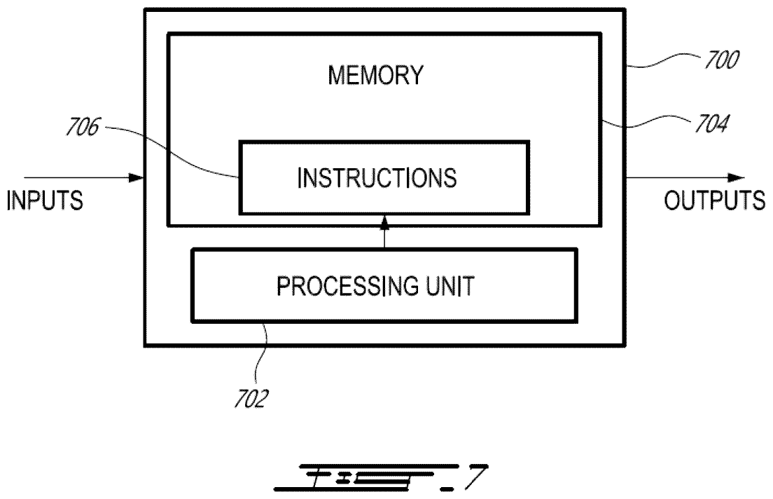

FIG. 7 illustrates an example computing device 700, such as the controller 200, which may be used to implement the system 100 of FIG. 1 and/or the methods 400 and/or 600 of FIGS. 5 and 6, respectively. The computing device 700 comprises a processing unit 702 and a memory 704 which has stored therein computer-executable instructions 706. The processing unit 702 may comprise any suitable devices configured to implement the functionality of the system 100 and/or the methods 500 and/or 600 such that instructions 706, when executed by the computing device 700 or other programmable apparatus, may cause the functions/acts/steps performed by the system 100 and/or the methods 500 and/or 600 as described herein to be executed. The processing unit 702 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, custom-designed analog and/or digital circuits, or any combination thereof.

The memory 704 may comprise any suitable known or other machine-readable storage medium. The memory 704 may comprise non-transitory computer readable storage medium, for example, but not limited to, an electronic, magnetic, optical, electromagnetic, infrared, or semiconductor system, apparatus, or device, or any suitable combination of the foregoing. The memory 704 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 704 may comprise any storage means (e.g., devices) suitable for retrievably storing machine-readable instructions 706 executable by processing unit 702.

The computing device 700 may be any suitable computing device, such as a desktop computer, a laptop computer, a mainframe, a server, a distributed computing system, a portable computing device, a mobile phone, a tablet, or the like. The following discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “communicatively connected” or “communicatively coupled to” may include both direct coupling (in which two elements that are coupled to each other contact each other) and indirect coupling (in which at least one additional element is located between the two elements). For instance, the term “communicatively connected” or “communicatively coupled to” may include a wireless connection over a communication network such as the Internet.

While illustrated in the block diagrams as groups of discrete components communicating with each other via distinct data signal connections, it will be understood by those skilled in the art that the preferred embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present preferred embodiment. The embodiments of the technology described above are intended to be exemplary only. For instance, as knee or lumbar surgeries are described above, they are meant to be exemplary only. The methods and systems described herein can also be applicable in pelvis surgery, shoulder blade surgery, and any other bone or articulation surgery. Moreover, in some embodiments, the ultrasound methods and systems can be used to find tools, screws and other surgery equipment within the body of the patient during surgery. The scope of the invention is therefore intended to be limited solely by the scope of the appended claims.

1. An intraoperative anatomical imaging system comprising:
a support panel having a support surface configured for supporting a patient, the support surface defining a plurality of apertures distributed thereon;
at least one peg having an extremity insertable in one of the apertures such that when the extremity is inserted in the aperture the at least one peg is secured to the support surface, the at least one peg configured to position the patient on the support surface;
at least one ultrasound probe unit; and
a controller being communicatively coupled to the ultrasound probe, the controller having a processor and a memory having stored thereon instructions that when executed by the processor perform the step of:
controlling the at least one ultrasound probe unit to emit ultrasound signals towards an anatomical feature of the patient,
measuring echo signals returning from the anatomical feature, and
generating an ultrasound image of the anatomical feature based on the echo datasets;
wherein the at least one ultrasound probe unit is secured to at least one of the at least one peg and the support surface such that the ultrasound image of the anatomical feature is obtainable when the patient is positioned on the support surface.

2. The intraoperative anatomical imaging system of claim 1, wherein the ultrasound probe comprises a plurality of ultrasound probe units mounted to at least one of the support surface and the at least one peg.

3. The intraoperative anatomical imaging system of claim 1, wherein at least one of the support surface and the at least one peg is configured to propagate the ultrasound signals and the echo signals.

4. The intraoperative anatomical imaging system of claim 1, wherein the ultrasound probe unit is integrated inside at least one of the at least one peg.

5. The intraoperative anatomical imaging system of claim 1, wherein the ultrasound probe unit is on an underside of the support surface.

6. The intraoperative anatomical imaging system of claim 1, wherein generating an ultrasound image of the anatomical feature includes generating an anatomical feature model of the anatomical feature based at least on the echo datasets.

7. The intraoperative anatomical imaging system of claim 6, wherein said generating the anatomical feature model includes merging the ultrasound image with a reference model of the anatomical feature.

8. The intraoperative anatomical imaging system of claim 7, wherein said reference model is obtained from a magnetic resonance imaging scan.

9. The intraoperative anatomical imaging system of claim 7, wherein said reference model is obtained from a bone atlas of models.

10. The intraoperative anatomical imaging system of claim 1, further comprising a coordinate tracking unit tracking coordinates of at least one of the support panel, the at least one peg and the ultrasound probe unit.

11. The intraoperative anatomical imaging system of claim 10, wherein the controller is communicatively coupled to the coordinate tracking unit and the memory has stored thereon instructions that when executed by the processor perform the steps of:
generating corresponding coordinate datasets,
registering the corresponding coordinate datasets in a common coordinate system, and
tracking a position and orientation of the anatomical feature based on said registering the echo datasets in the common coordinate system.

12. The intraoperative anatomical imaging system of claim 1, wherein the at least one ultrasound probe unit includes a first ultrasound probe unit configured to emit ultrasound signals towards the anatomical feature of the patient, and a second ultrasound probe unit for measuring echo signals returning from the anatomical feature.

13. An intraoperative anatomical imaging system comprising:
a support panel having a support surface configured for supporting a patient, the support surface defining a plurality of apertures distributed thereon;
at least one peg having an extremity insertable in one of the apertures such that when the extremity is inserted in the aperture the at least one peg is secured to the support surface, the at least one peg configured to position the patient on the support surface; and
at least one ultrasound probe unit secured to the at least one peg the ultrasound probe unit configured for emitting ultrasound signals towards different portions of an anatomical feature of the patient, and a second ultrasound probe unit for measuring echo signals returning from the portions of the anatomical feature, whereby an ultrasound image of the anatomical feature is obtainable when the patient is positioned on the support surface.

14. The intraoperative anatomical imaging system of claim 13, wherein the at least one ultrasound probe unit is integrated inside at least one of the at least one peg.

15. The intraoperative anatomical imaging system of claim 13, wherein the at least one ultrasound probe unit includes another ultrasound probe unit on an underside of the support surface.

16. The intraoperative anatomical imaging system of claim 13, wherein the at least one ultrasound probe unit includes a first ultrasound probe unit configured to emit ultrasound signals towards the anatomical feature of the patient, and a second ultrasound probe unit for measuring echo signals returning from the anatomical feature.

17. An intraoperative anatomical imaging system comprising:
a processor; and
a memory having stored thereon instructions that when executed by the processor perform the step of:
emitting ultrasound signals towards an anatomical feature of the patient, from a peg projecting from a support panel, with the anatomical feature captive against the peg and support panel,
measuring echo signals returning from the anatomical feature, and
generating and outputting an ultrasound image of the anatomical feature based on the echo datasets.

18. The intraoperative anatomical imaging system of claim 17, wherein generating an ultrasound image of the anatomical feature includes generating an anatomical feature model of the anatomical feature based at least on the echo datasets.

19. The intraoperative anatomical imaging system of claim 18, wherein said generating the anatomical feature model includes merging the ultrasound image with a reference model of the anatomical feature.

20. The intraoperative anatomical imaging system of claim 19, wherein said reference model is obtained from a magnetic resonance imaging scan.
